(19) United States
(12) Patent Application Publication
(10) Pub. No.: US 2025/0315100 A1
(43) Pub. Date: Oct. 9, 2025

(54) INFORMATION PROCESSING METHOD, INFORMATION PROCESSING DEVICE, INFORMATION PROCESSING RECORDING MEDIUM, METHOD FOR GENERATING MACHINE LEARNING TRAINED MODEL, AND MACHINE LEARNING TRAINED MODEL

(71) Applicant: OMRON HEALTHCARE Co., Ltd., Kyoto (JP)

(72) Inventors: Asami MATSUMURA, Kyoto (JP); Satoe NAGATA, Kyoto (JP); Yoko KANEMITSU, Kyoto (JP); Hironori SATO, Kyoto (JP); Yuji TAKAGI, Kyoto (JP); Miho INOUE, Kyoto (JP); Jeff RAY, Hoffman Estates, IL (US)

(21) Appl. No.: 19/244,733

(22) Filed: Jun. 20, 2025

Related U.S. Application Data
(63) Continuation of application No. PCT/JP2023/040267, filed on Nov. 8, 2023.

(30) Foreign Application Priority Data
Mar. 10, 2023 (JP) 2023-037755

(51) Int. Cl. G06F 3/01

(52) U.S. Cl. CPC G06F 3/011

(57) ABSTRACT

An information processing method, an information processing device, an information processing recording medium, a method for generating a machine learning trained model, and a machine learning trained model, which can be used for management of a measurement subject of biological information. A processor acquires measurement-related information related to a result of measurement of biological information performed on a measurement subject for a predetermined period by a biological information measuring device, derives measurement tendency information indicating a level of a possibility that the measurement subject continuously measures the biological information in a future period after the predetermined period based on the measurement-related information, and performs processing based on the measurement tendency information.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/040267, filed Nov. 8, 2023, which application claims priority to Japanese Patent Application No. 2023-037755, filed Mar. 10, 2023, which applications are incorporated herein by reference in their entireties.

### TECHNICAL FIELD

The present invention relates to an information processing method, an information processing device, an information processing recording medium, a method for generating a machine learning trained model, and a machine learning trained model.

### BACKGROUND

For health management, it may be required to continuously measure biological information such as weight, blood pressure, or blood glucose. However, some measurement subjects are likely to forget to measure the biological information. Patent Document 1 describes a technique in which measurement data of biological information of a user is acquired, and the user's piece in Sugoroku (Japanese board game) is moved forward based on the acquired data, whereby the user may be highly motivated to measure the biological information.

### CITATION LIST

### Patent Literature

Patent Document 1: JP 2019-3569 A

### SUMMARY OF INVENTION

### Technical Problem

Medical professionals such as physicians and nurses need to manage patients in such a manner that the patients that the medical professionals take care of continuously measure biological information. However, it is difficult for a medical professional who does not act together with a patient to determine whether the patient continuously measures his or her biological information.

An object of the present disclosure is to provide an information processing method, an information processing device, an information processing recording medium, a method for generating a machine learning trained model, and a machine learning trained model, which can be used for the management of a measurement subject of biological information.

### Solution to Problem

The technique of the present disclosure is as follows. Note that components and the like according to the following embodiments are indicated in parentheses, but the components are not limited thereto.
(1) An information processing method for causing a processor (a processor 11) to execute a process, the process including:
acquiring measurement-related information related to a result of measurement of biological information performed on a measurement subject for a predetermined period by a biological information measuring device;
deriving measurement tendency information indicating a level of a possibility that the measurement subject continuously measures the biological information in a future period after the predetermined period based on the measurement-related information; and,
performing processing based on the measurement tendency information.

According to (1), in a case that the measurement-related information of the measurement subject is acquired, it is possible to judge the level of the possibility that the measurement subject continues the biological information measurement in the future period. As a result, in a case that the possibility of continuation of the biological information measurement is judged to be low, it is possible to take measures such as prompting the measurement subject to measure the biological information, and raise the possibility that the measurement subject continuously measures the biological information.
(2) The information processing method according to (1), wherein the processor inputs the measurement-related information to a machine learning trained model and acquires the measurement tendency information from the model.

According to (2), by periodically subjecting the machine learning trained model to learning, derivation accuracy of the measurement tendency information may be improved and the measurement subject may be more appropriately managed.
(3) The information processing method according to (2), wherein the result of the measurement includes a measurement value of the biological information.

According to (3), since the measurement tendency information of the measurement subject can be derived by collecting the measurement values having been measured from the measurement subject, it is possible to easily derive the measurement tendency information without making the measurement subject perform special work.
(4) The information processing method according to (3), wherein the measurement-related information includes a representative value of the measurement values in the predetermined period.

According to (4), since the measurement tendency information of the measurement subject can be derived by collecting the measurement values having been measured from the measurement subject, it is possible to easily derive the measurement tendency information without making the measurement subject perform special work.
(5) The information processing method according to (3), wherein the measurement-related information includes information indicating a variation tendency of the measurement values in the predetermined period.

According to (5), since the measurement tendency information of the measurement subject can be derived by collecting the measurement values having been measured from the measurement subject, it is possible to easily derive the measurement tendency information without making the measurement subject perform special work.
(6) The information processing method according to (2), wherein the result of the measurement includes a measurement timing of the biological information in the predetermined period, and the measurement-related information includes information indicating features of distribution of the measurement timing.

According to (6), since the measurement tendency information of the measurement subject can be derived by collecting the measurement timings at which the biological information has been measured from the measurement subject, it is possible to easily derive the measurement tendency information without making the measurement subject perform special work.
(7) The information processing method according to (6), wherein the information includes an elapsed time from a timing at which the biological information is measured last in the predetermined period to a reference time point.

According to (7), since the measurement tendency information of the measurement subject can be derived by collecting the measurement timings at which the biological information has been measured from the measurement subject, it is possible to easily derive the measurement tendency information without making the measurement subject perform special work.
(8) The information processing method according to (6), wherein the information includes the number of times of measurement of the biological information in a period which is part of the predetermined period and is from the reference time point to a time point before a prescribed time.

According to (8), since the measurement tendency information of the measurement subject can be derived by collecting the measurement timings at which the biological information has been measured from the measurement subject, it is possible to easily derive the measurement tendency information without making the measurement subject perform special work.
(9) The information processing method according to any one of (2) to (8), wherein the processor is configured to acquire measurer information related to living conditions of the measurement subject, and further input the measurer information to the model to obtain the measurement tendency information from the model.

According to (9), since the measurement tendency information of the measurement subject can be derived using the measurer information and the measurement-related information of the measurement subject, it is possible to improve the derivation accuracy of the measurement tendency information.
(10) The information processing method according to (9), wherein the measurer information includes a timing at which the measurement subject lastly visited the hospital in the predetermined period, presence or absence of a person living together with the measurement subject, or the name of a disease from which the measurement subject is suffering.

According to (10), since the measurement tendency information can be derived in accordance with the living conditions of the measurement subject, it is possible to improve the derivation accuracy of the measurement tendency information.
(11) The information processing method according to any one of (2) to (8), wherein the processor is configured to acquire device-model information of the biological information measuring device used by the measurement subject, and further input the device-model information to the model to obtain the measurement tendency information from the model.

According to (11), since the measurement tendency information can be derived in accordance with the device-model of the biological information measuring device used by the measurement subject, it is possible to improve the derivation accuracy of the measurement tendency information.
(12) The information processing method according to any one of (2) to (11), wherein the model is generated by learning, as data for learning, the measurement-related information related to a result of measurement of biological information performed on the measurement subject for a predetermined period and intervention information indicating whether intervention for prompting the measurement subject to measure the biological information has been performed after the predetermined period, and the processor is configured to input, to the model, the acquired measurement-related information and intervention presence information indicating that intervention has been performed, and perform the processing based on the measurement tendency information acquired from the model, and input, to the model, the acquired measurement-related information and intervention absence information indicating that no intervention has been performed, and perform the processing based on the measurement tendency information acquired from the model.

According to (12), it is possible to, for example, compare the measurement tendency information when the intervention is present and the measurement tendency information when the intervention is absent, and this comparison makes it possible to support the determination of whether the intervention should be performed on the measurement subject.
(13) The information processing method according to (12), wherein the intervention information included in the data for learning and indicating that the intervention has been performed includes information of a time zone in which the intervention has been performed, and the processor performs processing of inputting, to the model, the acquired measurement-related information and information for performing intervention in a specified time zone a plurality of times while changing the time zone, and performs the processing based on the measurement tendency information output from the model in the processing having been performed the plurality of times.

According to (13), it is possible to compare, for example, the measurement tendency information for each time zone in which the intervention is performed with each other, and this comparison makes it possible to determine which time zone is effective for the intervention when the intervention is performed on the measurement subject.
(14) The information processing method according to (12), wherein the intervention information included in the data for learning and indicating that the intervention has been performed includes information of contents of the intervention having been performed, and the processor performs processing of inputting, to the model, the acquired measurement-related information and the information of the contents of the intervention a plurality of times while changing the information of the contents, and performs the processing based on the measurement tendency information output from the model in the processing having been performed the plurality of times.

According to (14), it is possible to, for example, compare the measurement tendency information for each content of the intervention with each other, and this comparison makes it possible to determine what content of the intervention is effective when the intervention is performed on the measurement subject.
(15) An information processing device (an information processing server 10) including a processor (a processor 11) that is configured to acquire measurement-related information related to a result of measurement of biological information performed on a measurement subject for a predetermined period by a biological information measuring device, derive measurement tendency information indicating a level of a possibility that the measurement subject continuously measures the biological information in a future period after the predetermined period based on the measurement-related information, and perform processing based on the measurement tendency information.
(16) An information processing recording medium for causing a processor (a processor 11) to execute a process, the process including:
a step of acquiring measurement-related information related to a result of measurement of biological information performed on a measurement subject for a predetermined period by a biological information measuring device;
a step of deriving measurement tendency information indicating a level of a possibility that the measurement subject continuously measures the biological information in a future period after the predetermined period based on the measurement-related information; and,
a step of performing processing based on the measurement tendency information.
(17) A method for generating a machine learning trained model, the method causing a processor (a processor 11) to:
acquire, as data for learning, a plurality of pieces of measurement-related information related to a result of measurement of biological information in a predetermined period (a period T1) in a constant period in which the measurement of the biological information was performed in the past on a measurement subject by a biological information measuring device, and a plurality of pieces of information regarding whether the measurement subject continuously measured the biological information in a period (a period T2) after the predetermined period in the constant period; and,
make a recording medium execute machine learning based on a plurality of pieces of the data for learning, and generate, when the measurement-related information related to the result of the measurement of the biological information performed on the measurement subject for the predetermined period by the biological information measuring device is input, a machine learning trained model (a machine learning trained model 13) that outputs measurement tendency information indicating a level of a possibility that the measurement subject continuously measures the biological information in a future period after the predetermined period.
(18) A machine learning trained model (a machine learning trained model 13) having been subjected to machine learning while taking, as data for learning, measurement-related information related to a result of measurement of biological information in a predetermined period (a period T1) in a constant period in which the measurement of the biological information was performed in the past on a measurement subject by a biological information measuring device, and information regarding whether the measurement subject continuously measured the biological information in a period (a period T2) after the predetermined period in the constant period, the machine learning trained model (machine learning trained model 13) causing a processor (a processor 11) to execute processing to output, while taking the measurement-related information related to the result of the measurement of the biological information performed on the measurement subject by the biological information measuring device for the predetermined period as input, measurement tendency information indicating a level of a possibility that the measurement subject continuously measures the biological information in a future period after the predetermined period.

### Advantageous Effects of Invention

According to the present disclosure, it is possible to assist the management of a measurement subject of biological information.

### BRIEF DESCRIPTION OF DRAWINGS

FIG. 1 is a schematic diagram illustrating a schematic configuration of a management system 100.

FIG. 2 is a diagram schematically illustrating measurement data.

FIG. 3 is a flowchart for explaining a method for generating a machine learning trained model.

FIG. 4 is a diagram schematically illustrating measurement data of a user X.

FIG. 5 is a diagram illustrating a modified example of the management system 100.

FIG. 6 is a diagram illustrating an example of a screen displayed on a display device of a facility terminal 40.

FIG. 7 is a diagram illustrating another example of a screen displayed on the display device of the facility terminal 40.

### DESCRIPTION OF EMBODIMENTS

### Outline of Information Processing Method of Present Disclosure

An information processing method is a method for causing a processor to acquire measurement-related information related to a result of measurement of biological information performed on a measurement subject for a predetermined period by a biological information measuring device such as a weight scale, a blood pressure monitor, or a blood glucose measuring instrument; derive measurement tendency information indicating a level of a possibility that the measurement subject continuously measures the biological information in a future period after the predetermined period based on the measurement-related information; and perform processing based on the measurement tendency information.

As the measurement-related information, information indicating the magnitude of the measurement value or the tendency of a change in the measurement value of the biological information in the predetermined period, or information indicating the features of distribution of the measurement timings of the biological information in the predetermined period is preferably used. As the measurement tendency information, information of a probability that the measurement subject continues the biological information measurement in a future period is preferably used. In this way, by predicting how long the measurement subject will continue to measure the biological information in the future based on the result of the measurement of the biological information by the measurement subject, it is possible to give an appropriate advice or the like to the measurement subject and make the measurement subject continue to measure the biological information.

Hereinafter, a configuration example of a management system including a device configured to execute the information processing method of the present disclosure will be described.

### System Configuration

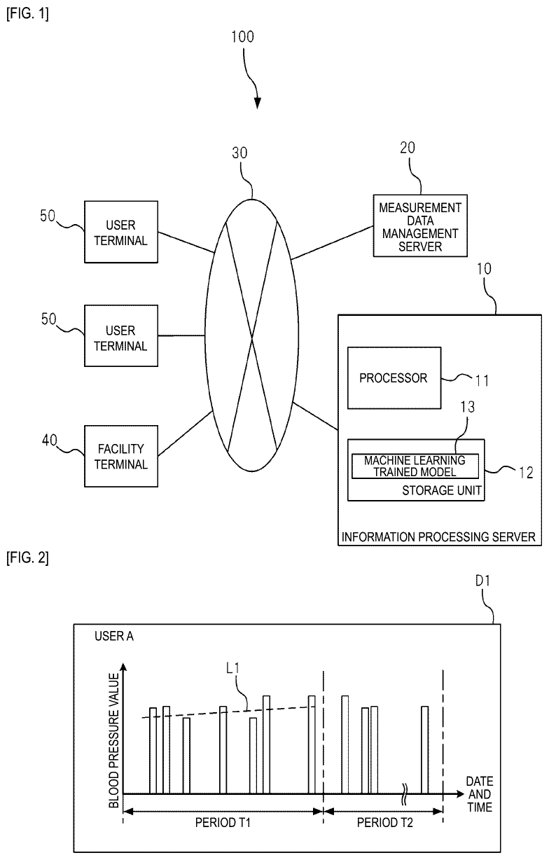

FIG. 1 is a schematic diagram illustrating a schematic configuration of a management system 100. The management system 100 is a system for supporting a person (hereinafter referred to as a user) who needs management of biological information such as a weight, a blood pressure, a pulse, or blood glucose so that the user can continuously measure the biological information. The management system 100 includes an information processing server 10, a measurement data management server 20, a facility terminal 40, and a plurality of user terminals 50, and these are configured to be connectable to a network 30 such as the Internet.

The user terminal 50 is an electronic device such as a smartphone carried by a user. A biological information measuring device such as a weight scale, a blood pressure monitor, a pulsimeter, or a blood glucose measuring instrument carried by the user and the user terminal 50 are communicably connected to each other, and measurement data measured by the biological information measuring device is transmitted from the user terminal 50 to the measurement data management server 20. The measurement data includes a measurement value of biological information such as a weight, a blood pressure value, a pulse rate, or a blood glucose level, and information of a measurement date and time. Hereinafter, an example will be described in which the biological information measuring device is a blood pressure monitor and the measurement value is a blood pressure value (preferably, a systolic blood pressure).

The measurement data management server 20 stores the measurement data transmitted from the user terminal 50 in a database in association with information for identifying the user, and manages the measurement data for each user. FIG. 2 is a diagram schematically illustrating measurement data. FIG. 2 illustrates measurement data D1 of a user A.

The measurement data includes a plurality of sets of measurement date and time and a measurement value (blood pressure value) measured at the measurement date and time. In the example of FIG. 2, the magnitude of the systolic blood pressure as the measurement value is depicted in the form of a bar graph. The date and time with no bar graph represents date and time when no measurement was performed. The measurement data described above is collected from a large number of users over a long period of time and accumulated in the database.

The measurement data management server 20 includes a sample data group from which training data used to generate a machine learning trained model 13 described later is extracted. Each piece of the measurement data included in the sample data group includes, for example, a measurement result (including measurement timings and measured values) in a predetermined period T1 starting from a day on which a usage registration of the user is made with respect to the measurement data management server 20, and a measurement result in a predetermined period T2 starting from a day next to the final day of the period T1. Although the period T1 and the period T2 are not particularly limited, the period T2 is set to be longer than the period T1. As an example, the period T1 is 30 days and the period T2 is 90 days.

The facility terminal 40 is an electronic device, such as a personal computer, a smartphone, or a tablet terminal, installed in a medical facility such as a hospital. By accessing the measurement data management server 20 from the facility terminal 40, the measurement data of a specific user can be downloaded to the facility terminal 40 and referenced. The facility terminal 40 includes, for example, a display device such as an organic electro-luminescence (EL) display or a liquid crystal display, or a speaker.

The information processing server 10 includes a processor 11 and a storage unit 12. The storage unit 12 is configured to include, for example, a non-transitory storage medium such as a hard disk or flash memory in addition to a working memory such as a random access memory (RAM). The storage unit 12 stores an information processing recording medium for the information processing server 10 to execute the information processing method.

The processor 11 is, for example, a central processing unit (CPU) that is a general-purpose processor executing software (recording medium) to perform various functions, a programmable logic device (PLD) that is a processor whose circuit configuration can be changed after manufacturing, such as a field programmable gate array (FPGA), or a dedicated electric circuit that is a processor having a circuit configuration dedicatedly designed to execute specific processing, such as an application specific integrated circuit (ASIC). The processor 11 may be configured with one processor, or may be configured with a combination of two or more processors of the same type or different types (for example, a plurality of FPGAs or a combination of a CPU and an FPGA). More specifically, the hardware structure of the processor 11 is an electric circuit (circuitry) in which circuit elements such as semiconductor elements are combined. When the processor 11 is configured with a plurality of processors, the plurality of processors do not need to be installed within the same device, and may be installed in each of a plurality of devices dispersedly disposed via a network 30.

The storage unit 12 stores the machine learning trained model 13. The machine learning trained model 13 is generated by causing a learning model configured by a recording medium to execute machine learning using training data. The machine learning trained model 13 may be generated by the processor 11 of the information processing server 10, or may be generated by a processor of a computer different from the information processing server 10. Hereinafter, a method for generating the machine learning trained model 13 will be described assuming that the processor 11 generates the machine learning trained model 13.

### Method for Generating Machine Learning Trained Model

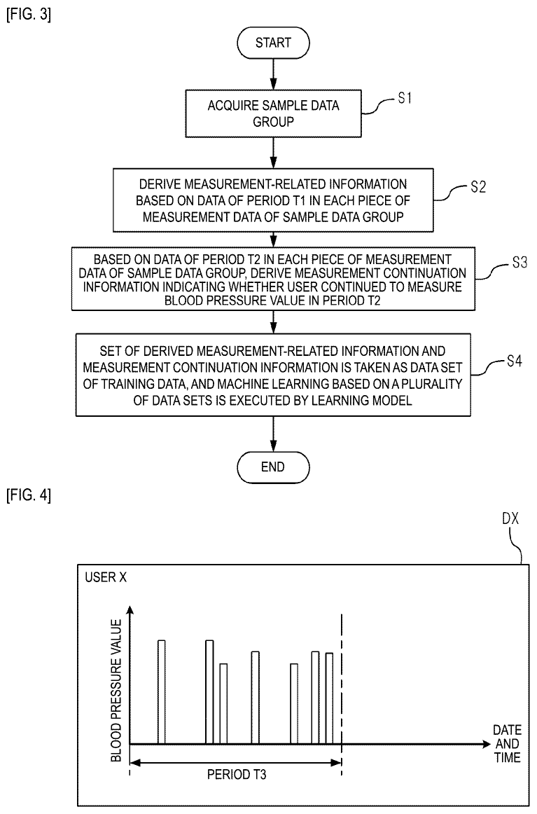

FIG. 3 is a flowchart for explaining a method for generating a machine learning trained model.

The processor 11 acquires the above-described sample data group from the measurement data management server 20 (step S1). Subsequently, the processor 11 derives, based on the data of a period T1 in each piece of the measurement data of the acquired sample data group, measurement-related information related to the measurement result (in other words, information indicating features of the measurement result) of the blood pressure value in the period T1 (step S2).

The measurement-related information includes, for example, first information indicating features of the measurement value in the period T1, or second information indicating features of the distribution of the measurement timings in the period T1.

The first information is, for example, a representative value of the blood pressure values in the period T1. The representative value is a mean value of the blood pressure values measured in the period T1, a mean value of the blood pressure values measured in the period T1 excluding the minimum and maximum values, a median value of the blood pressure values measured in the period T1, or the like.

As another example, the first information is information indicating a variation tendency of the blood pressure value in the period T1. For example, when a straight line L1 indicating a time-series change in the blood pressure value in the period T1 of the measurement data D1 depicted in FIG. 2 is derived by the least squares method, a slope of the straight line L1 is the information indicating the variation tendency. The first information may be an image of the graph depicted in FIG. 2, in which a change in the blood pressure value in the period T1 can be seen.

The second information is, for example, information indicating whether the number of times of measurement is smaller in a period close to the period T2 in the period T1. For example, when the period T1 is divided into a plurality of groups, the number of times of measurement of the blood pressure value in a group closest to the period T2 (a period from the final day of the period T1 to a time point before a prescribed time) among the plurality of groups can be used as the second information. As another example, an elapsed time from the date and time when the blood pressure value is measured last in the period T1 to the last day of the period T1 can be used as the second information.

Subsequently, the processor 11 derives, based on the data of the period T2 in each piece of the measurement data of the acquired sample data group, measurement continuation information indicating whether the user continued to measure the blood pressure value in the period T2 (step S3). The expression “the user continues to measure the blood pressure value” means that the number of times of measurement of the blood pressure value in the period T2 is equal to or greater than a predetermined value (e.g., three times). The measurement continuation information is information indicating the measurement being continued (e.g., “1” or “True”) when the number of times of measurement of the blood pressure value in the period T2 is equal to or greater than the predetermined value, and is information indicating the measurement being not continued (e.g., “0” or “False”) when the number of times of measurement of the blood pressure value in the period T2 is less than the predetermined value.

The processor 11 takes the measurement-related information and the measurement continuation information having been derived based on each piece of the measurement data as a data set of the training data, and causes the learning model to execute machine learning based on a plurality of the data sets to generate the machine learning trained model 13.

The machine learning trained model 13 has learned various parameters in such a manner that, when the measurement-related information related to the measurement result of the blood pressure values for a predetermined period (a period having the same length as the period T1) performed on the user by a blood pressure monitor is input, the measurement tendency information indicating a level of a possibility that the user continuously measures the blood pressure value in a future period (a period having the same length as the period T2) after the predetermined period is output. The measurement tendency information is preferably information indicating a probability that the user continuously measures the blood pressure value (or a probability that the user does not continuously measure the blood pressure value) in the future period.

As described above, the machine learning trained model 13 is a model in which machine learning has been performed to estimate and output a probability that the user continues the measurement in a future period with respect to the measurement-related information obtained from the past measurement data of a specific user based on an enormous amount of the measurement-related information and the measurement continuation information corresponding thereto. The machine learning method is not particularly limited. For example, any of methods such as logistic regression, a decision tree, random forests, a gradient boosting decision tree, and a neural network can be used.

According to a result of statistical analysis of an enormous amount of measurement data acquired in the past, a user who tends to have a high blood pressure value in the period T1 (a user having a large representative value of the blood pressure values) tends not to continue the measurement of the blood pressure value in the subsequent period T2. It may be said that this is caused by a decrease in motivation to continue the measurement due to the continuously measured blood pressure value being high. Further, according to the above result, a user whose blood pressure value is likely to increase in the period T1 tends not to continue the measurement of the blood pressure value in the subsequent period T2. It may be said that this is caused by a decrease in motivation to continue the measurement because the continuously measured blood pressure value tends not to be improved but worsened.

According to the above-described result, in a period close to the period T2 in the period T1 (for example, when the period T1 is divided into a first half and a second half, the above period corresponds to the second half), a user with a small number of times of measurement tends not to continue the measurement of the blood pressure value in the subsequent period T2. Further, according to the above-described result, a user who takes a long time from the last measurement timing in the period T1 to the end of the period T1 (a user who has not performed measurement for a while recently) tends not to continue the measurement of the blood pressure value in the subsequent period T2. These ideas can be similarly applied to other biological information such as a weight and a blood glucose level.

Thus, by the set of the measurement-related information and the measurement continuation information being subjected to machine learning, it is possible to generate a model for estimating a possibility that the user continues the measurement of the blood pressure value in the future period from the measurement-related information obtained from the measurement data of the specific user.

### Use of Machine Learning Trained Model

The processor 11 uses the machine learning trained model 13 generated as described above to estimate a level of a possibility that the user who has acquired only measurement data for a period T3 having the same length as the period T1 continues the measurement of the blood pressure value in a future period (a period having the same length as the period T2) after the period T3, and performs processing based on the estimation result.

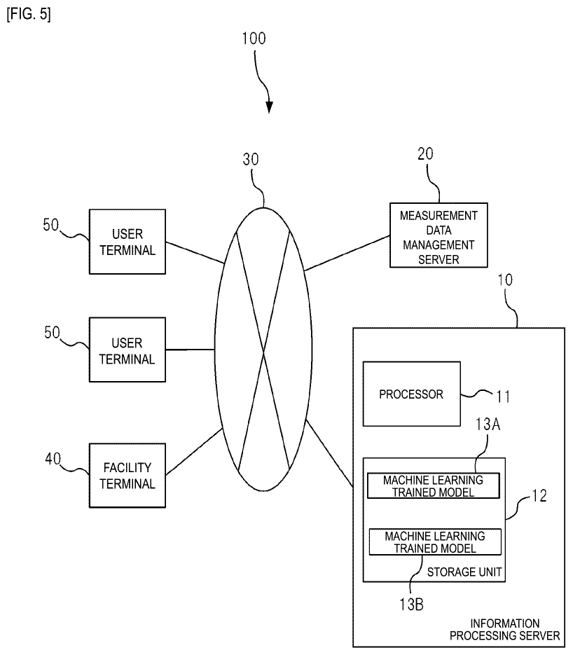

For example, as illustrated in FIG. 4, it is assumed that a medical professional wants to know whether a specific user X, whose measurement data DX for the period T3 has been obtained, will continue the measurement in a future period after the period T3. The medical professional operates the facility terminal 40 to read the measurement data DX of the user X at a time point of the final day of the period T3, transmits the read measurement data DX to the information processing server 10, and requests the information processing server 10 to estimate a measurement continuation probability of the user X in the future period after the final day.

When the processor 11 of the information processing server 10 receives the measurement data DX, the processor 11 derives measurement-related information related to the measurement result in the period T3 based on the measurement data DX. For example, the processor 11 derives a representative value (such as a mean value or a median value) of the blood pressure values in the period T3 of the measurement data DX as the measurement-related information. A processor of the facility terminal 40 may be configured to derive the measurement-related information based on the measurement data DX. In this case, the processor of the facility terminal 40 transmits the derived measurement-related information to the information processing server 10 to request the estimation of the measurement continuation probability.

Subsequently, the processor 11 inputs the measurement-related information derived by the processor 11 itself or the measurement-related information received from the facility terminal 40 to the machine learning trained model 13, and acquires, from the machine learning trained model 13, measurement tendency information indicating a level of a possibility that the user X continues the measurement of the blood pressure value (preferably, probability information) in a future period after the period T3.

When the processor 11 acquires the measurement tendency information, the processor 11 performs processing for displaying the measurement tendency information on the display device of the facility terminal 40 or outputting the measurement tendency information by sound from the speaker of the facility terminal 40. The processor 11 may transmit the measurement tendency information to the facility terminal 40 in the form of an electronic mail or the like.

For example, by checking the measurement tendency information displayed on the display device of the facility terminal 40, the medical professional can determine whether the user X tends to continue the measurement of the blood pressure value from now on. For example, in a case where the measurement tendency information (the probability of continuing the measurement) is low, the medical professional actively performs intervention (notification using an application installed in the user terminal 50 carried by the user X, transmission of an electronic mail, telephone, or the like) for prompting the user X to measure the blood pressure value. This makes it possible to raise the possibility that the user X continuously measures the blood pressure value. Since such intervention can be appropriately performed, a rise in health awareness of the user X himself or herself, an improvement in diagnosis accuracy because of being able to determine a change in the blood pressure value of the user X in detail, and the like can be expected, which can contribute to health promotion of the user X.

Each piece of the measurement data of the sample data group for generating the machine learning trained model 13 includes the measurement date and time and the measurement values. However, when the second information (information indicating features of the distribution of the measurement timings) is used as the measurement-related information to be input to the machine learning trained model 13, each piece of the measurement data may include at least the measurement date and time and may be allowed not to include the measurement values.

### Modified Example of Machine Learning Trained Model

Although the machine learning trained model 13 is obtained by performing machine learning while using the measurement-related information and the measurement continuation information as the training data, machine learning may be performed by further including measurer information in the training data. The measurer information is information regarding living conditions of the user as the measurement source of each piece of the measurement data of the sample data group. The measurer information is, for example, information of a timing at which the user lastly visited the hospital in the period T1, information of presence or absence of a person living together with the user, or information of the name of a disease from which the user is suffering. In this case, the measurer information regarding the living conditions of the user as the acquisition source of each piece of the measurement data is further added to each piece of the measurement data of the sample data group stored in the database by the measurement data management server 20.

The processor 11 sets the information obtained by combining the measurer information included in each piece of the measurement data of the sample data group and the measurement-related information obtained from each piece of the measurement data to be training data, sets the measurement continuation information obtained from each piece of the measurement data to be training data, and causes the learning recording medium to execute machine learning based on the training data set to generate the machine learning trained model 13. Thus, when the set of the measurer information and the measurement-related information is input, it is possible to generate the machine learning trained model 13 configured to output measurement tendency information indicating a level of a possibility that the user corresponding to the set continues the measurement in a future period.

In this way, by further using the measurer information of the user, it is possible to more accurately estimate whether the user continues the measurement of the biological information in the future period. For example, between a user who has visited the hospital in the period T1 and a user who has not visited the hospital in the period T1, the health awareness of the former is higher than that of the latter, and therefore the former has a higher possibility of continuing the measurement. In addition, between a user living together with another person and a user not living together with another person, the former is more likely to continue the measurement because of the existence of the person who may point out the measurement being forgotten and the like. By learning the tendency of the measurement continuation based on the living conditions discussed above, it is possible to accurately estimate the level of the possibility that the user continues the measurement.

Instead of the measurer information described above, device-model information of a biological information measuring device used by the user to measure the biological information may be used. The tendency of the measurement continuation may also change depending on usability (ease of communication, ease of measurement, and the like) of the biological information measuring device. Therefore, by learning the tendency depending on the device-model in use as discussed above, it is possible to more accurately estimate the level of the possibility that the user continues the measurement.

### First Modified Example of Management System

FIG. 5 is a diagram illustrating a modified example of the management system 100. The management system 100 illustrated in FIG. 5 is different from that illustrated in FIG. 1 in that the machine learning trained model stored in the storage unit 12 is changed to two machine learning trained models 13A and 13B.

In the management system 100 illustrated in FIG. 5, as sample data groups stored in a database by the measurement data management server 20, there are an intervention presence sample data group including the measurement data of a user who has been prompted by intervention to measure the blood pressure in the period T2, and an intervention absence sample data group including the measurement data of a user who has not been prompted by intervention to measure the blood pressure in the period T2. Information indicating the presence of intervention (e.g., “1”) is associated with each piece of the measurement data of the intervention presence sample data group. Information indicating the absence of intervention (e.g., “0”) is associated with each piece of the measurement data of the intervention absence sample data group.

The machine learning trained model 13A is a model generated by performing machine learning on the training data set of the measurement-related information and the measurement continuation information generated based on each piece of the measurement data of the intervention presence sample data group, and constitutes a first model.

The machine learning trained model 13B is a model generated by performing machine learning on the training data set of the measurement-related information and the measurement continuation information generated based on each piece of the measurement data of the intervention absence sample data group, and constitutes a second model.

In the management system 100 illustrated in FIG. 5, for example, the processor 11 inputs the measurement-related information derived based on the measurement data DX of the user X illustrated in FIG. 4 to each of the machine learning trained model 13A and the machine learning trained model 13B, and performs processing based on the measurement tendency information output from each of the machine learning trained model 13A and the machine learning trained model 13B.

For example, the processor 11 causes the display device of the facility terminal 40 to display the measurement tendency information output from the machine learning trained model 13A and the measurement tendency information output from the machine learning trained model 13B together, causes the display device of the facility terminal 40 to display a difference between the two pieces of the measurement tendency information (probabilities) as an effect of performing the intervention, or the like. Specifically, the display device is caused to display a message such as “A probability that the user X continues the measurement in the future is 30%, but the probability can be raised to 60% by performing intervention”. As described above, since the effect of the intervention is clearly indicated, the medical professional can be free from uncertainty in determining whether or not to perform intervene on the user X.

The storage unit 12 may prepare and store, as the machine learning trained model 13A, a plurality of types of models in which intervention has been performed in different time zones. For example, as the machine learning trained model 13A, a morning intervention model and an afternoon intervention model are generated. The morning intervention model is generated by performing machine learning on a training data set of the measurement-related information and the measurement continuation information generated based on each piece of the measurement data of the sample data group of the user on which intervention has been performed in the morning (e.g., from nine to noon). The afternoon intervention model is generated by performing machine learning on a training data set of the measurement-related information and the measurement continuation information generated based on each piece of the measurement data of the sample data group of the user on which intervention has been performed in the afternoon (e.g., from noon to five).

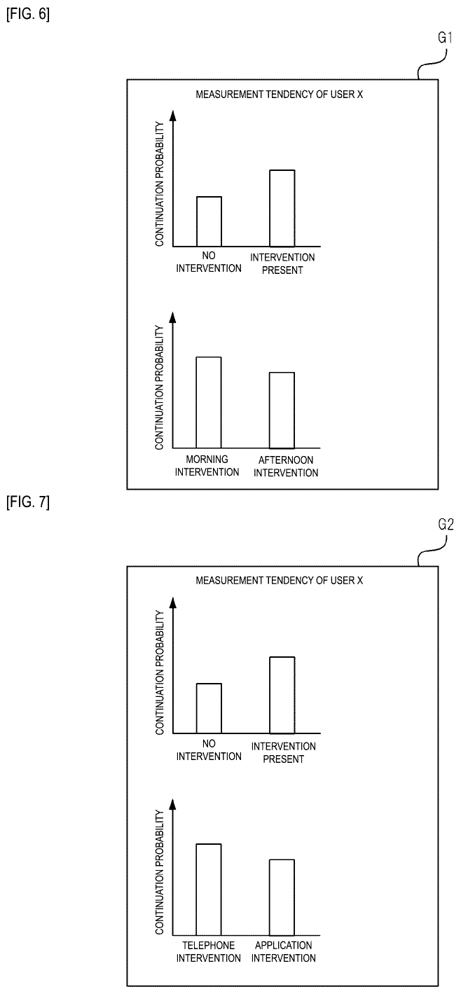

Then, for example, the processor 11 inputs the measurement-related information derived based on the measurement data DX of the user X depicted in FIG. 4 to each of the morning intervention model of the machine learning trained model 13A, the afternoon intervention model of the machine learning trained model 13A, and the machine learning trained model 13B, and performs processing based on measurement tendency information output from each model (for example, processing for displaying a screen G1 illustrated in FIG. 6). This processing makes it possible to know not only a change in the probability of continuing the measurement depending on the presence or absence of the intervention, but also which of the intervention performed in the morning and the intervention performed in the afternoon raises the probability. For example, it is possible to further raise the possibility that the user continues the blood pressure measurement by performing intervention on the user in a time zone (in the morning in the example of FIG. 6) in which the effect is most enhanced.

The storage unit 12 may prepare and store, as the machine learning trained model 13A, a plurality of types of models with interventions of different contents. For example, as the machine learning trained model 13A, a telephone intervention model and an application intervention model are generated. The telephone intervention model is generated by performing machine learning on a training data set of the measurement-related information and the measurement continuation information generated based on each piece of the measurement data of the sample data group of the user on which intervention by telephone has been performed. The application intervention model is generated by performing machine learning on a training data set of the measurement-related information and the measurement continuation information generated based on each piece of the measurement data of the sample data group of the user on which intervention by application notification has been performed.

Then, for example, the processor 11 inputs the measurement-related information derived based on the measurement data DX of the user X depicted in FIG. 4 to each of the telephone intervention model of the machine learning trained model 13A, the application intervention model of the machine learning trained model 13A, and the machine learning trained model 13B, and performs processing based on measurement tendency information output from each model (for example, processing for displaying a screen G2 illustrated in FIG. 7). This processing makes it possible to know not only a change in the continuation probability depending on the presence or absence of intervention but also how the probability changes depending on the content of the intervention when the intervention is performed. For example, it is possible to further raise the possibility that the user continues the blood pressure measurement by performing intervention on the user by the content (telephone in the example of FIG. 7) that most enhances the effect. The content of the intervention may include the content of a message transmitted to the user at the time of the intervention.

### Second Modified Example of Management System

The machine learning trained model 13A and the machine learning trained model 13B may be achieved by one machine learning trained model (referred to as a machine learning trained model 13C). That is, the machine learning trained model 13C may be a model generated by performing machine learning on a training data set of measurement-related information generated based on each piece of the measurement data of the sample data group, intervention information indicating the presence or absence of intervention associated with the measurement data, and measurement continuation information generated based on the measurement data.

For example, the processor 11 derives measurement-related information based on the measurement data DX of the user X depicted in FIG. 4, inputs the measurement-related information and information indicating the presence of intervention to the machine learning trained model 13C, acquires first measurement tendency information (measurement continuation tendency in a case of intervention in the future) output from the machine learning trained model 13C, inputs the measurement-related information and information indicating the absence of intervention to the machine learning trained model 13C, acquires second measurement tendency information (measurement continuation tendency in a case of no intervention in the future) output from the machine learning trained model 13C, and performs processing based on the first measurement tendency information and the second measurement tendency information.

For example, the processor 11 causes the display device of the facility terminal 40 to display the first measurement tendency information and the second measurement tendency information together (see the upper graph in each of FIG. 6 and FIG. 7), causes the display device of the facility terminal 40 to display a difference between the first measurement tendency information and the second measurement tendency information (probabilities) as an effect of performing the intervention, and the like.

Of the intervention information in the training data set used in the second modified example, the information indicating the presence of intervention may be further added with information of the time zone in which the intervention has been performed (hereinafter referred to as intervention time zone information).

In this case, for example, the processor 11 derives measurement-related information based on the measurement data DX of the user X depicted in FIG. 4, inputs the measurement-related information and information indicating the presence of morning intervention to the machine learning trained model 13C, acquires third measurement tendency information (measurement tendency in a case of morning intervention in the future) output from the machine learning trained model 13C, inputs the measurement-related information and information indicating the presence of afternoon intervention to the machine learning trained model 13C, acquires fourth measurement tendency information (measurement tendency in a case of afternoon intervention in the future) output from the machine learning trained model 13C, and performs processing based on the third measurement tendency information and the fourth measurement tendency information.

For example, the processor 11 causes the display device of the facility terminal 40 to display the third measurement tendency information and the fourth measurement tendency information together (see the lower graph in FIG. 6), causes the display device of the facility terminal 40 to display a difference between the third measurement tendency information and the fourth measurement tendency information (probabilities) as an effect depending on a difference in the intervention time zone, and the like.

Instead of the intervention time zone information mentioned above, information of the content of the performed intervention (intervention content information) may be used. In this case, for example, the processor 11 derives measurement-related information based on the measurement data DX of the user X illustrated in FIG. 4, and performs processing of inputting the measurement-related information and information indicating a specific intervention content to the machine learning trained model 13C a plurality of times while changing the intervention content. The processor 11 acquires a plurality of pieces of measurement tendency information (measurement tendency for each intervention content in a case where intervention is performed in the future) output from the machine learning trained model 13C through the plurality of times of processing, and performs processing based on the plurality of pieces of measurement tendency information.

For example, the processor 11 causes the display device of the facility terminal 40 to display the plurality of pieces of measurement tendency information together (see the lower graph in FIG. 7), causes the display device of the facility terminal 40 to display a difference between the plurality of pieces of measurement tendency information (probabilities) as an effect depending on a difference in the intervention content, and the like.

In the above description, the processor 11 derives the measurement tendency information from the measurement-related information using the machine learning trained model. However, a table storing the correspondence between the measurement-related information and the measurement tendency information may be generated and stored in the storage unit 12, and the processor 11 may derive future measurement tendency information of the user X based on the measurement-related information derived based on the measurement data of the user X and the table.

For example, the measurement data of the sample data group is divided into a plurality of groups by the magnitude of the representative value of the blood pressure values in the period T1, and (the number of users having continued the measurement/the total number of users in the group) is obtained as a continuation probability in each group from the measurement continuation information corresponding to the measurement data belonging to each group. The above-discussed table may be generated by associating the continuation probability obtained in this manner with the group. When the processor 11 acquires the measurement-related information of the user X, the processor 11 may specify the group to which the measurement-related information belongs and acquire the continuation probability corresponding to the specified group from the table. In the case where a machine learning trained model is used as discussed above, machine learning is performed every time sample data is accumulated, thereby making it possible to enhance the estimation accuracy of the continuation probability by the model. For this reason, it is more preferable to use the machine learning trained model.

Although various embodiments have been described above, it is needless to say that the present invention is not limited to such examples. It will be apparent to those skilled in the art that various changes and modifications can be made within the scope of the claims, and it is understood that these naturally belong within the technical scope of the present invention. Further, components of the above-described embodiments may be combined as desired within a range that does not depart from the spirit of the present invention.

### REFERENCE NUMERALS LIST

D1 Measurement data
T1, T2, T3 Period
L1 Straight line
10 Information processing server
11 Processor
12 Storage unit
13, 13A, 13B Machine learning trained model
20 Measurement data management server
30 Network
40 Facility terminal
50 User terminal
100 Management system

1. An information processing method for causing a processor to execute a process, the process comprising:
acquiring measurement-related information related to a result of measurement of biological information performed on a measurement subject for a predetermined period by a biological information measuring device;
inputting the measurement-related information to a machine learning trained model, and deriving, from the model, measurement tendency information indicating a level of a possibility that the measurement subject continuously measures the biological information in a future period after the predetermined period; and
performing processing based on the measurement tendency information, wherein
the result of the measurement includes a measurement timing of the biological information in the predetermined period, and
the measurement-related information includes information indicating features of distribution of the measurement timing.

2. The information processing method according to claim 1, wherein
the result of the measurement includes a measurement value of the biological information.

3. The information processing method according to claim 1, wherein
the processor is configured to
acquire measurer information related to living conditions of the measurement subject, and
further input the measurer information to the model to obtain the measurement tendency information from the model.

4. The information processing method according to claim 1, wherein
the processor is configured to
acquire device-model information of the biological information measuring device used by the measurement subject, and
further input the device-model information to the model to obtain the measurement tendency information from the model.

5. The information processing method according to claim 1, wherein
the model is generated by learning, as data for learning, the measurement-related information related to the result of the measurement of the biological information performed on the measurement subject for a predetermined period and intervention information indicating whether intervention for prompting the measurement subject to measure the biological information has been performed after the predetermined period, and
the processor is configured to
input, to the model, the acquired measurement-related information and intervention presence information indicating that intervention has been performed, and perform the processing based on the measurement tendency information acquired from the model, and
input, to the model, the acquired measurement-related information and intervention absence information indicating that no intervention has been performed, and perform the processing based on the measurement tendency information acquired from the model.

6. The information processing method according to claim 5, wherein
the intervention information included in the data for learning and indicating that the intervention has been performed includes information of a time zone in which the intervention has been performed, and
the processor performs processing of inputting, to the model, the acquired measurement-related information and information for performing intervention in a specified time zone a plurality of times while changing the time zone, and performs the processing based on the measurement tendency information output from the model in the processing having been performed the plurality of times.

7. The information processing method according to claim 5, wherein
the intervention information included in the data for learning and indicating that the intervention has been performed includes information of contents of the intervention having been performed, and
the processor performs processing of inputting, to the model, the acquired measurement-related information and the information of the contents of the intervention a plurality of times while changing the information of the contents, and performs the processing based on the measurement tendency information output from the model in the processing having been performed the plurality of times.

8. An information processing device, comprising a processor that is configured to
acquire measurement-related information related to a result of measurement of biological information performed on a measurement subject for a predetermined period by a biological information measuring device,
input the measurement-related information to a machine learning trained model, and derive, from the model, measurement tendency information indicating a level of a possibility that the measurement subject continuously measures the biological information in a future period after the predetermined period, and perform processing based on the measurement tendency information, wherein
the result of the measurement includes a measurement timing of the biological information in the predetermined period, and
the measurement-related information includes information indicating features of distribution of the measurement timing.

9. An information processing recording medium for causing a processor to execute a process, the process comprising:
acquiring measurement-related information related to a result of measurement of biological information performed on a measurement subject for a predetermined period by a biological information measuring device;
inputting the measurement-related information to a machine learning trained model, and deriving, from the model, measurement tendency information indicating a level of a possibility that the measurement subject continuously measures the biological information in a future period after the predetermined period; and
performing processing based on the measurement tendency information, wherein
the result of the measurement includes a measurement timing of the biological information in the predetermined period, and
the measurement-related information includes information indicating features of distribution of the measurement timing.

10. A method for generating a machine learning trained model, the method causing a processor to
acquire, as data for learning, a plurality of pieces of measurement-related information related to a result of measurement of biological information in a predetermined period in a constant period in which the measurement of the biological information was performed in the past on a measurement subject by a biological information measuring device, and a plurality of pieces of information regarding whether the measurement subject continuously measured the biological information in a period after the predetermined period in the constant period, and
make a recording medium execute machine learning based on a plurality of pieces of the data for learning, and generate, when the measurement-related information related to the result of the measurement of the biological information performed on the measurement subject for the predetermined period by the biological information measuring device is input, a machine learning trained model that outputs measurement tendency information indicating a level of a possibility that the measurement subject continuously measures the biological information in a future period after the predetermined period, wherein
the result of the measurement includes a measurement timing of the biological information in the predetermined period, and
the measurement-related information includes information indicating features of distribution of the measurement timing.

11. A machine learning trained model having been subjected to machine learning while taking, as data for learning, measurement-related information related to a result of measurement of biological information in a predetermined period in a constant period in which the measurement of the biological information was performed in the past on a measurement subject by a biological information measuring device, and information regarding whether the measurement subject continuously measured the biological information in a period after the predetermined period in the constant period, the machine learning trained model causing
a processor to execute processing to output, while taking the measurement-related information related to the result of the measurement of the biological information performed on the measurement subject for the predetermined period by the biological information measuring device as input, measurement tendency information indicating a level of a possibility that the measurement subject continuously measures the biological information in a future period after the predetermined period, wherein
the result of the measurement includes a measurement timing of the biological information in the predetermined period, and
the measurement-related information includes information indicating features of distribution of the measurement timing.
